(19) United States
(12) Patent Application Publication
(10) Pub. No.: US 2007/0141158 A1
(43) Pub. Date: Jun. 21, 2007

(54) Amphiphilic heparin derivative formed by coupling a heparin with a bile acid

(75) Inventors: Didier Hoarau, Quebec (CA); Mahfoud Boustta, Pignan (FR); Christian Braud, Castelnau Le Lez (FR); Michel Vert, Castelnau Le Lez (FR)

Correspondence Address: FOLEY AND LARDNER LLP;SUITE 500, 3000 K STREET NW, WASHINGTON, DC, 20007, US

(73) Assignee: ETHYPHARM, HOUDAN (FR)

(21) Appl. No.: 10/582,989

(22) PCT Filed: Dec. 17, 2004
(86) PCT No.: PCT/FR04/03285

§ 371 Date: Jan. 10, 2007

(30) Foreign Application Priority Data
Dec. 19, 2003 (FR) 0315003

(51) Int. Cl. A61K 31/727; C08B 37/10; A61K 9/14

(52) U.S. Cl. 424/489; 514/56; 536/21; 977/906

(57) ABSTRACT

The invention relates to an amphiphilic heparin derivative formed from at least one type of partially N-desulfated heparin and at least one type of bile acid comprising one or several bile acid molecules grafted on a heparin molecule by an amide bond formed between the terminal carboxylic acid function of a bile acid and a primary heparin amine function which is initially present in the heparin or resulting from the N-desulfation. The inventive derivative is characterized in that the number of grafted bile acid molecules per 100 heparin disaccharide units ranges from 15 to 80 approximately.

The present invention relates to an amphiphilic heparin derivative formed by coupling heparin with a bile acid, having the capacity to form nanoparticles spontaneously in an aqueous medium.

The nanoparticles formed from this heparin derivative may be used as a vector for active ingredients for administration by the oral route.

They make it possible in particular to solubilize and transport hydrophobic active ingredients across the intestinal mucous until they come into close contact with the intestinal mucosa, to release said active ingredients gradually and to promote absorption thereof.

The present invention additionally relates to the method for synthesizing this amphiphilic heparin derivative.

The present invention finally relates to the uses which may be made of such a vector, in particular in the therapeutic field for the administration of active ingredients by the oral route, and more particularly as vector for active ingredients weakly absorbed by the intestinal mucosa. The present invention also relates to the use of said vector according to the invention for the transport of active ingredients in combination with a conventional galenic carrier used for the administration of active ingredients by the oral route, such as granules, microgranules, gelatin capsules and solutions to be taken orally in particular.

The expression hydrophobic is understood to mean any compound insoluble in water or with very little affinity for water, and the expression amphiphilic is understood to mean any compound having at the same time some affinity for the aqueous phases and some affinity for the organic phases.

In addition, the name nanoparticle denotes any particle less than or equal to one micrometer in size, and the name microparticle denotes any particle of between 1 and 1000 μm in size.

The expression promotion of absorption is understood to mean any means capable of being used in order to increase the quantity of active ingredient which crosses the intestinal mucosa.

The mucus is a clear and ropy product of normal secretion of the mucous glands of which mucin is the main constituent and which contains water, salts and desquamated cells. It plays a protective role toward the mucous membranes which it covers both from a mechanical point of view and from a chemical point of view.

Bile acids are cholesterol derivatives produced by the liver and secreted into the bile where they are stored. They make it possible to maintain cholesterol in soluble form. When the bile reaches the small intestine after a meal, these bile acids will promote the solubilization, homogenization, emulsification (dissolution) and absorption of the fats derived from foods.

Primary bile acids (for example cholic acid and chenodeoxycholic acid) and secondary bile acids (for example deoxycholic acid and lithocholic acid) are distinguishable.

Finally, the expression bioavailability is understood to mean the fraction of molecule of active ingredient found in the bloodstream after administration by the oral route. Bioavailability is measured by evaluating the plasma level of active ingredient observed compared with the plasma level of this same active ingredient administered by the intravenous route.

A large portion of the therapeutic active ingredients administered by the oral route is absorbed in the small intestine, and more particularly in the upper part of the small intestine: the duodenum. This absorption involves, in a first instance, the passage of the molecule of active ingredient across the plasma membrane of the cells of the intestinal epithelium, the enterocytes, and then the crossing of the vascular endothelium of the blood vessels.

Such an absorption depends on numerous parameters which influence the efficacy of absorption of the active ingredient considered and more particularly the crossing of the intestinal mucosa. However, three main parameters should be considered in order to explain the low bioavailability of certain active ingredients administered by the oral route.

First of all, the solubility of the active ingredient in the gastrointestinal medium is sometimes reduced, or even zero for some active ingredients of a particularly hydrophobic nature.

Indeed, it is possible to consider the gastrointestinal medium as a mainly aqueous medium since it is partly composed of chyme and partly of gastric juices. As a result, the oral administration of these active ingredients is not possible if they have not been subjected beforehand to a very fine dispersion or to dissolution in an organic phase consisting of the assembly of the hydrophobic parts of a micelle of surfactants or of the core of a nanoparticle.

Moreover, some active ingredients, although water-soluble, are the subject of an “absorption window”, that is to say that their absorption is only possible in a defined area of the intestine, such as the duodenum for example. Here again, the combination of these active ingredients with a transporter or vector, which exhibits affinity for the intestinal mucosa, makes it possible to increase the residence time of these compounds in an environment close to their site of absorption.

Finally, the active ingredient should have, in the gastrointestinal and therefore aqueous medium, an external structure which allows it to approach the intestinal membrane in order for it to be absorbed by the enterocytes. Indeed, the epithelial cells of the intestine and more particularly of the duodenum are covered with a dense mucus consisting of an entanglement of glycoproteins. This molecular network constitutes a physical barrier of the hydrophilic gel type which the molecules of hydrophobic active ingredients must cross in order to be in contact with the plasma membrane of the enterocytes (Larhed et al. 1997). Thus, the molecule of active ingredient, alone or in combination with a vector, must have a physical configuration allowing it to approach this membrane. In particular, two types of constraints exist at this level: size constraints and load constraints. Indeed, the active ingredient or the vector/active ingredient complex must have a sufficiently small size (less than a micrometer) allowing it to diffuse into the network of glycooproteins. In addition, some authors like Park and Robinson (1984) have observed that the phenomenon of bioadhesion to the gastrointestinal mucus was improved for molecules having surface charges, compared with uncharged molecules.

The preparation of vectors for active ingredients weakly absorbed by the intestinal mucosa should therefore make it possible to reduce the influence of these parameters in order to promote the crossing of the intestinal barrier by the active ingredient and therefore the bioavailability of these compounds.

Thus, such vectors should be able to not only transport the active ingredients to their specific site for intestinal absorption, but also to promote their solubility in the gastrointestinal medium and/or increase their intestinal transit time.

The present invention therefore aims to provide a novel type of vector intended to improve the absorption of active ingredients exhibiting low bioavailability by the oral route, that is to say which are hardly absorbed or not absorbed by the intestinal mucosa.

Among all the existing vectors, the use of vectors in the form of microparticles or nanoparticles loaded with active ingredients makes it possible to envisage the transport of weakly soluble compounds.

Thus, vesicles of the liposome type have been developed with the aim of transporting such compounds.

These nanoparticles consist of a membrane composed of a double layer of phospholipids, whose inner and outer surfaces are hydrophilic, which membrane delimits an aqueous compartment within. The liposomes thus make it possible to transport water-soluble active ingredients incorporated within this central cavity. The document WO 9308802 describes for example liposomes intended to transport and to release active ingredients of the type including tricyclic immuno-suppressants. Such liposomes allow a stable transport of these active ingredients in various aqueous media and in particular physiological fluids, while maintaining the stability of the active ingredients in solutions for injection such as glucose solutions for example.

However, this type of vector has two major disadvantages. First of all, the stability of the liposomes in the digestive medium is low. Indeed, these structures can be easily destabilized by surfactants such as bile salts. Moreover, the lipids constituting these vectors are rapidly degraded, in particular by the digestive enzymes, such as lipases, which reduces the efficiency of absorption of the active ingredient.

Patent JP58049311 discloses a means for resolving the problem of instability of the liposomes by grafting fatty acid esters of polysaccharides at their surface. These polysaccharide-coated liposomes then have a markedly increased plasma stability and therefore a longer life in the bloodstream.

Moreover, the document JP 61268628 allows the targeting of the active ingredient onto its site of absorption by grafting at the surface of the transporting liposomes polysaccharides onto which are attached monoclonal antibodies specific for the desired site of absorption. The use, by the oral route, of these liposomes stabilized by polysaccharides esterified with fatty acids has not been envisaged. However, this type of liposomal vector has the disadvantage of being able to transport only molecules of a predominantly hydrophilic nature, and therefore of being scarcely suitable for the transport of lipophilic molecules which are of course scarcely soluble in an aqueous medium. Moreover, their use in the gastrointestinal fluids is impossible because of their sensitivity to the intestinal lipases and to the bile salts. Finally, the targeting of the absorption site requires a complex step of grafting of specific antibodies. This step makes the method for synthesizing these liposomes long and delicate since it is necessary beforehand to produce and then couple said antibodies to the polysaccharides before attaching them to the surface of the liposomes.

The work carried out on the modified saccharide polymers used as liposome stabilizers has made it possible to discover that such hydrophobized polysaccharides had the capacity to self-aggregate in the form of clusters having a hydrophobic core, consisting of the assembly of grafted hydrophobic units, and a hydrophilic surface, consisting of the backbone of the saccharide polymer (Akiyoshi et al. 1993).

Thus, vector systems for active ingredients on this model have been used. Patent WO 9842755 indeed describes vectors for medicaments consisting of vesicles formed from polysaccharide derivatives. These Chitosan, Pullulan or Dextran derivatives contain at least one monosaccharide residue substituted with a nonionic hydrophilic compound and at least one mono-saccharide residue substituted with a hydrophobic group. This hydrophobic group is of the type including alkyl, alkenyl, alkynyl or acyl with a long chain. The formation of the vesicles is then induced in the presence of cholesterol. Because of the hydrophobization of the hydrophilic polymer backbone, the complex precipitates in water and requires the presence of cholesterol and/or a steric stabilizer in order to obtain the vector in vesicular form. In this model, the vesicular formation is induced, which involves an additional formulation step in order to trigger the passage in particulate form.

On the other hand, in the document U.S. Pat. No. 4,997,819, a hydrophobized polysaccharide is used as stabilizer for emulsions of fatty acids. The fatty vesicles formed are used with the aim of encapsulating various lipophilic substances and in particular active ingredients. The polysaccharide preferably used in this case is Pullulan, and the hydrophobic groups used to hydrophobize this polymer are cholesterol or certain fatty acids. The complex called CHP for the abbreviation of Cholesterol-Pullulan is the complex preferably used. The number of hydrophobic groups grafted onto the polysaccharide is variable. In this document, the modified polymer is not the predominant component at the origin of the lipid particles formed, it only constitutes a stabilizer thereof.

Other work on molecules related to CHPs has led to the use of these multimolecular clusters as vectors for active ingredients. Indeed, these complexes form spontaneously in an aqueous medium and exist in the form of nanoparticles having several inner hydrophobic domains into which a compound of a hydrophobic nature can be introduced.

Patent JP 7097333 thus describes a supramolecular complex composed of a hydrophobized polysaccharide intended to serve as transporter for certain cytokines. This complex corresponds to the combination of residues of the sterol type, preferably cholesterol, with a polysaccharide molecule, preferably Pullulan. The grafting of the hydrophobic residue occurs in this case via a spacer arm. This type of transporter is used to increase the plasma lifespan of these active ingredients administered by the intramuscular or percutaneous route.

Thus, these vesicles make it possible to encapsulate fat-soluble active ingredients dissolved in their hydrophobic inner domains. These hydrophobized polymers are therefore of great interest for the administration of such compounds.

However, such types of vectors do not allow the transport of active ingredients to their site of absorption on the intestinal mucosa.

Moreover, such vectors do not allow improvement in the intestinal absorption of the active ingredients which they transport.

Another saccharide polymer, heparin, is capable of forming aggregates in an aqueous medium if certain hydrophobic residues are grafted onto its saccharide chain. It is possible for example to couple heparin to dodecanol, to cholic acid or to stearic acid after partial N-desulfation (F. Diancourt et al.). The study shows that in this hydrophobized form, heparin preserves its full biological activity, in particular its anticoagulant activity after administration by the intravenous route or by the subcutaneous route. However, in this study, heparin is not used as a vector for active ingredients, but as active ingredient itself. The objective of this study is to cause heparin to cross the intestinal mucosa and to check if it preserves its anticoagulant properties after having been hydrophobized with various hydrophobic residues.

The aim of the present invention is to provide a novel type of vector for active ingredients which are normally weakly or poorly absorbed by the intestinal mucosa, said vector making it possible to specifically increase this absorption by transporting the active ingredients across the intestinal mucus, and then gradually releasing them in close contact with the intestinal wall, but without the vector crossing this wall. The vector according to the invention is also designed to solubilize the active ingredients of a hydrophobic nature, while being of simple design, including only constituent elements derived from digestible metabolites or prometabolites and being capable of being easily loaded with active ingredient.

The present invention aims to provide, moreover, a nontoxic and biocompatible vector for active ingredients having the capacity to spontaneously go into an aqueous medium in the form of nanoparticles having a mean diameter of less than one micrometer. Said nanoparticles are at the same time stable over time, a lot more stable than liposomes and other amphiphilic micellar combinations, including the micelles of diblock amphiphilic copolymers, and capable of gradually releasing their contents in contact with the intestinal membrane. This capacity to closely approach the intestinal membrane being due to their particular amphiphilic structure combining charged constituent elements and lipophilic elements allows them to easily diffuse within the network of glycoproteins which the intestinal mucus constitutes.

Moreover, the nanoparticles according to the invention have the advantage of significantly increasing the residence time of the active ingredient transported since they interact favorably with the glycoproteins of the intestinal mucus or even become destabilized therein in order to release the active agent locally following interactions with the macromolecules constituting the mucus. This interaction resulting from a prolonged phenomenon of mucoadhesion promotes the absorption of the active ingredients, in particular for those which have an absorption “window”.

It is thus possible to envisage in particular the transport of medicaments having a certain hydrophobic character, such as those belonging to the class of anti-inflammatory agents, antifungal agents, calcium channel inhibitors or anticancer agents for example. More particularly, it may be possible to provide for the transport of active ingredients such as nifedipine, progesterone, carbamazepine or itraconazole.

In addition, the present invention has the advantage of being simple to carry out and inexpensive, being composed exclusively of natural molecules that are easily available and assembled with each other by simple chemical reactions.

Finally, the vector for active ingredients in accordance with the invention may be easily combined or incorporated into a conventional galenic carrier used for the oral administration of medicaments. Indeed, it is possible to allow for incorporation into tablets, granules, or alternatively powder, gelatin capsules or solution to be taken orally, of the nanoparticles in accordance with the invention, loaded beforehand with the active ingredient considered and purified. Such a vector can therefore enter simply into the composition of medicaments intended to be administered by the oral route.

The small-sized particles (micro- and nanoparticles) arriving in contact with the intestinal mucosa will have three types of outcome, closely linked to their size and to their chemical nature. These particles will first of all be captured by the lymphoid tissue associated with the intestine, according to a phenomenon of endocytosis at the level of Peyer's patch cells. Moreover, these particles can also be retained at the level of the mucus by a phenomenon of mucoadhesion. Finally, these particles may be directly removed in fecal materials.

Several authors have thus observed that particles greater than one micrometer in size were easily removed during the intestinal transit because of their low level of penetration into the intestinal mucus (Ponchel et al. 1997). The mucous network is indeed too dense for particles of such a size to be able to diffuse therein.

Moreover, the endocytosis phenomenon observed in the lymphoid tissue is a phenomenon which affects more particularly the particles having a surface that is not charged and is of a hydrophobic nature (Desai et al. 1996; O'Hagan 1996; Florence 1997). The extent of this endocytosis phenomenon is however minimal given that the Peyer's patches represent only a minute surface of the total surface of the intestinal mucosa.

The vector for active ingredients in accordance with the invention is made by the spontaneous assembly in an aqueous medium of several heparin molecules onto which at least one hydrophobic residue derived from at least one bile acid has been grafted so as to produce a polymer of an amphiphilic nature.

This polymer makes it possible to promote to the maximum the phenomenon of mucoadhesion of the nanoparticles formed in accordance with the present invention while creating a vector capable of transporting a lipophilic active ingredient in dissolved form. Thus, the vector for active ingredients in accordance with the invention satisfies several criteria of size and structure which allow it to be particularly suited to the environment of the intestine.

The subject of the present invention is an amphiphilic heparin derivative formed from an at least partially N-desulfated heparin and from at least one bile acid, comprising one or more bile acid molecules grafted onto the heparin molecule by an amide bond formed between the terminal carboxylic acid functional group of the bile acid and a primary amine functional group of the heparin, originally present in the heparin or resulting from the N-desulfation, in which the number of bile acid molecules grafted per 100 disaccharide units of the heparin is between about 15 and about 80, preferably between about 20 and about 60.

Heparin is a complex macromolecule consisting of an assembly of saccharide units of the class of glycosaminoglycans. The polymer chain predominantly consists of an acidic sugar (uronic acid) and an amino sugar (glucosamine) arranged in a regular alternating pattern. The corresponding disaccharide unit is multi-sulfated in well-defined positions. The acid functional groups are in the carboxylate and sulfate form. The three main units of the heparin may be repeated in the following manner:

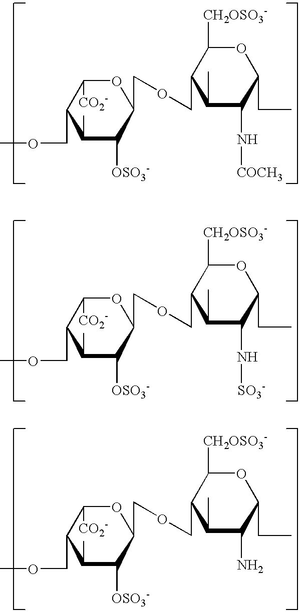

The nitrogen of the amino sugar is essentially in the N-sulfate form (in more than 80% of the units), but may also be in the N-acetyl form (about 15% of the units); the units comprising the free amine form are very poorly represented (1 to 2% at the chain end). The amine functional groups which are mainly in the N-sulfate form are not available for the chemical coupling reactions. It is therefore preferable to have heparin having numerous primary amine functional groups to which the coupling of hydrophobic groups may be carried out. In the present invention, this involves an amidation reaction: the carboxylic acid functional group of the bile acid will have to react with the amine functional group of the polymer in order to form an amide bond.

Heparin therefore constitutes a polyelectrolyte which is essentially negatively charged by sulfate (O—SO₃⁻ or NH—SO₃⁻) or carboxylate (COO⁻) groups in the natural state.

The bile acid is advantageously chosen from cholic acid, deoxycholic acid, lithocholic acid, cholanic acid and chenodeoxycholic acid, and mixtures thereof.

For example, cholic acid has the following chemical formula:

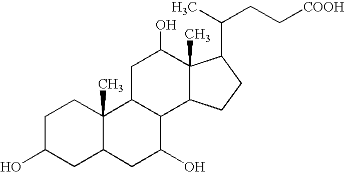

The cholic acid which enters into the composition of the bile salts is a steroid derived from cholesterol of a predominantly hydrophobic nature.

However, the hydroxyl functional groups carried by this residue confer a degree of hydrophilicity on the cholic acid molecule. The hydrophilic groups carried by the cholic acid reduce the strength of aggregation of the hydrophobic groups with each other. As a result, the lipophilic core of these supramolecular complexes will therefore be more loose than if it consisted solely of residues of cholesterol for example (which is a markedly less hydrophilic molecule). This phenomenon will contribute toward increasing the diameter of the nanoparticles formed by the assembly of these complexes, and also determine the affinity of the active ingredients for the hydrophobic domains.

The amphiphilic heparin derivative which is the subject of the present invention may be advantageously prepared in calcium, magnesium or sodium salt form.

The subject of the present invention is also the nanoparticles which can be formed from the amphiphilic heparin derivative as defined above.

The amphiphilic heparin derivatives in accordance with the invention indeed have the capacity to assemble in an aqueous medium in order to form a stable colloidal suspension of nanoparticles having a diameter of between 10 nanometers and 1 micrometer. The mean diameter observed is nevertheless particularly homogeneous, of the order of 300 nanometers.

The nanoparticles which are the subject of the present invention have a hydrophilic outer surface and one or more hydrophobic inner domains.

In aqueous medium, the hydrophobic residues of the polymer come close together and form noncovalent crosslinking points which are responsible for the formation of the amphiphilic nanoparticles, allowing the constitution of hydrophobic inner domains within which lipophilic active ingredients may be dissolved and therefore transported.

The heparins hydrophobized by the bile acid have a behavior in an aqueous solution which is different from that of the native heparin. The more hydrophobized the polymer, the less soluble it is in water. The aqueous solutions are opalescent and stable. These solutions become colored in orange after the addition of Yellow OB, a lipophilic marker which colors the solutions orange when it is solubilized in an organic phase. The orange color then makes it possible to prove the presence of hydrophobic domains in the molecule synthesized in accordance with the present invention, since this marker does not dissolve in water or in a solution of nonhydrophobized heparin.

The subject of the present invention is also said nanoparticles additionally containing one or more hydrophobic active ingredients dissolved in their hydrophobic inner domains.

Said active ingredients preferably carry one or more polar groups.

They are preferably chosen from anti-inflammatory agents, antifungal agents, calcium channel inhibitors and anticancer agents.

The subject of the present invention is also said nanoparticles as vectors for active ingredients which can be administered by the oral route.

The subject of the present invention is also said nanoparticles as vectors for active ingredients which make it possible to increase their absorption by the intestinal mucosa, and/or which allow their gradual release in the intestinal mucosa.

Indeed, the nanoparticles which are the subject of the present invention have the property of being able to reach and of course to remain in contact with or in an environment close to the intestinal membrane. The amphiphilic heparin derivative which is the subject of the present invention possesses all the qualities necessary for good diffusion within the mucus and for good mucoadhesion.

Thus, the charged groups carried by the vector in accordance with the invention which interact favorably with the groups carried by the glycoproteins of the mucus, make it possible to increase their transit time in contact with the intestinal mucosa by diffusing within the network of the glycoproteins of the mucus. This potentiation of the mucoadhesion phenomenon, by increasing the contact time between the active ingredient and the intestinal membrane, promotes its absorption.

The choice of heparin as polysaccharide backbone is therefore vital since this polymer has at the same time numerous ionized functional groups in an aqueous medium (polyelectrolyte with a high charge density) and also primary amine functional groups which can be easily released, making the coupling of the hydrophobic residue possible. The high charge density ensures the solubility of the system in the colloidal state following the coupling of numerous hydrophobic residues, avoiding its massive precipitation in the aqueous media. The formation of the nanoparticles occurs by spontaneous self-assembly in the aqueous media and does not require the addition of surfactants or of agents for steric stabilization. Finally, heparin was advantageously chosen because it constitutes a natural polymer that is absolutely well tolerated by the body, and is in fact commonly used, by the parenteral route, in therapy in humans as an anticoagulant.

The choice of a bile acid as hydrophobizing agent is also one of the essential characteristics of the invention since this natural compound will allow the modified polymer to assemble in the form of stable nanoparticles in the intestinal medium but also to produce intermolecular interactions which ensure not only the cohesion of the system but the solubilization of active ingredients of a hydrophobic nature. These noncovalent interactions subsequently make it possible to release the active ingredient content of the nanoparticles in the vicinity of the lipid membranes of the intestinal cells.

Thus, the combination of heparin with at least one bile acid allows the formation of nanoparticles that are sufficiently stable in the intestinal environment to remain intact until there is close contact with the intestinal mucosa. However, the nanoparticles in accordance with the invention are sufficiently labile and biodegradable to then gradually release the active ingredient which they contain into the mucous environment in the vicinity of the lipid membrane of the intestinal cells, without crossing the intestinal mucosa.

The nanoparticles which are the subject of the present invention have numerous advantageous properties in terms of size, stability and capacity for incorporation of active ingredients.

The subject of the present invention is also the colloidal suspension in aqueous medium containing said nanoparticles. This suspension may for example be used to prepare an oral suspension or alternatively may be sprayed onto neutral supports in order to prepare granules.

The subject of the present invention is also the pharmaceutical composition comprising said nanoparticles combined with at least one pharmaceutically acceptable excipient.

In this pharmaceutical composition, the excipient is advantageously chosen to allow administration of active ingredients by the oral route.

Said pharmaceutical composition may be provided in the form of granules, microgranules, tablets, gelatin capsules or solutions to be taken orally.

The subject of the present invention is also a method for preparing the amphiphilic heparin derivative, which comprises the at least partial N-desulfation of a heparin, and then a coupling step which consists in reacting at least one primary amine functional group of the heparin, originally present or resulting from the N-desulfation, with the terminal carboxylic acid functional group, optionally in activated form, of at least one bile acid.

The preparation of the nanoparticles may be followed by a freeze-drying step in order to be able to preserve them more easily.

The active ingredient may be incorporated into the nanoparticles by direct dissolution with stirring, by dialysis, by oil/water emulsion, by solvent evaporation.

Method of Preparation

As explained above, it is preferable to have available heparin having numerous primary amine functional groups to which the coupling of the hydrophobic groups may be carried out.

The release of the primary amine functional groups will occur by selective hydrolysis of the N-sulfate functional groups according to a method which makes it possible to accurately control the N-desulfation level.

Preferably, this step is followed by a step of formation of a cetyltrimethylammonium salt of the desulfated polysaccharide molecule, so as to confer solubility in organic medium on it, before applying the method of coupling the cholic acid residues to the amine functional groups released. This salt is then removed at the end of the coupling step.

a) N-Desulfation

The desulfation is preferably selective on the N-sulfate groups so as to not hydrolyze the O-sulfates, which would result in a decrease in the number of ionized groups and therefore a loss of solubility.

Two methods may be used: hydrolysis by auto-catalysis of heparinic acid which corresponds to the method traditionally used and hydrolysis in solvent medium or “solvolysis” of heparinic acid.

The main disadvantage of hydrolysis by auto-catalysis is the time required to obtain products having acceptable levels of N-desulfation. Indeed, such a reaction takes between one week and one month.

On the other hand, solvolysis makes it possible to obtain in a few hours heparin derivatives with a high proportion of primary amine functional groups. In particular, this method makes it possible, for fixed concentration and temperature parameters, to reproducibly obtain derivatives having the desired primary amine functional group content, by stopping the hydrolysis reaction at a given time.

According to this method, the selective hydrolysis of the N-sulfate groups may be obtained by placing heparinic acid salified with pyridine in a mixture of DMSO and water (or DMSO/methanol) in which the proportion of water does not exceed 10%. Under these conditions, it is possible to obtain partially or completely N-desulfated samples, without causing depolymerization or impairment of the structure (Nagasawa and Inoue 1974; Inoue and Nagasawa 1976). The speed of this reaction may be controlled by the temperature for a given heparin concentration. Thus, by stopping the reaction at various times, samples are obtained in a few hours with the desired levels of N-desulfation.

The disaccharide units of the heparin molecule are in the N-sulfate form for slightly more than 80% of them. Preferably, for carrying out the present invention, the heparin molecule is desulfated at a level between 10 and 65%. This level makes it possible to obtain a level of between 8 and 52% of the disaccharide units in the N-desulfated form, that is to say in the form appropriate for the step of coupling the hydrophobic residue, and more particularly the bile acid.

The N-desulfation may be carried out in the following manner.

The purification of the heparin is first of all carried out by dialysis (or ultrafiltration). Next, the heparin solution is percolated at 4° C. over a cation-exchange resin in H⁺ form. A solution of heparinic acid is then obtained. The concentration of the solution obtained is then adjusted such that the proportion of residual water represents 5% of the final total volume when the DMSO will be added. Thus, the heparinic acid solution may be freeze-dried after the concentration step or concentrated to dryness before adding the desired quantity of water. The solution is then transferred to a flask with a large volume for the reaction. A sufficient quantity of pyridine representing as many equivalents as acid functional groups is added to the solution obtained above. These functional groups are then in the form of a pyridinium salt. A volume of DMSO is then added until the following concentration is obtained: DMSO/H₂O (95/5 v/v). The heparin concentration in the solution is 2% (m/v). This solution is then preferably heated to 40° C., but different temperatures may also be used depending on the rate of hydrolysis which it is desired to have. Samples are collected at various times.

Each sample is placed in an ice bath and water is added thereto, with stirring (the reaction is indeed inhibited with a proportion of water greater than 25%). The medium is then neutralized with a sufficient quantity of sodium hydroxide to pH 7-8.

Two alternatives then exist according to whether it is desired to isolate the product obtained or to directly prepare a salt with cetyltrimethylammonium bromide:

In the Case of the Isolation of N-desulfated Heparin in the Form of a Sodium Salt:

The N-desulfated heparin is present in a solution comprising released sulfate ions, pyridinium salts, DMSO. The solution is dialyzed several times against water so as to remove these impurities, and then it is subjected to a concentration step and, finally, a freeze-drying step. Purified N-desulfated heparin is therefore obtained in dry form for which the percentage N-desulfation can then be determined.

In the Case of the Direct Production of the Quaternary Ammonium Salt:

After sample collection and neutralization, a cetyltrimethylammonium bromide solution is added. The latter compound forms an insoluble salt with heparin which precipitates in aqueous medium. It is then separated by filtration, and rinsed several times with hot water so as to remove the sulfate ions and the other water-soluble molecules. The product is then dried. It may be subsequently used for the coupling step.

The choice of temperature is made according to what it is desired to obtain and the control of the reaction. Thus, by using 20° C. it is easier to control the production of weakly N-desulfated heparins whereas at 40° C. the rate of hydrolysis is too high at the start, which is not suitable for recovering weakly N-desulfated heparins but rather for heparins having 20 to 60% N-desulfation. By using a temperature greater than or equal to 50° C., a completely N-desulfated sample is obtained in 24 hours.

Preferably, hydrophobized heparins having an N-desulfation level of between 8 and 65% will be used for the production of nanoparticles in accordance with the present invention and which are well suited to the transport of active ingredients.

b) Method of Assaying the Primary Amine Functional Groups of the Nonhydrophobized N-desulfated Heparin

This method is based on the calorimetric method of assaying amines developed by Snyder and Sobocinski (1975). The assay is based on the determination of the optical density at a wavelength of 420 nm for the chromophore formed by covalent bonding between 2,4,6-trinitrobenzenesulfonic acid (TNBS) and the free amine functional groups. Given that TNBS in basic medium is degraded progressively according to a 0 order linear kinetics and releases picric acid which interferes at 420 nm with the chromophore formed, blanks (without heparin) are also prepared. This reaction is specific for the NH₂ groups.

The Procedure may be the Following:
Preparation of N-desulfated heparin solutions, for example in accordance with the method described, in 0.1 M borate buffer at pH 10, and distribution of 500 μl in tubes.
Addition of 500 μl of a 0.08 M TNBS solution (diluted in distilled water) to all the tubes.
Collection of aliquots at various times. Dilution and measurement of the Optical Density (OD) at 420 nm.
Kinetic profiles of order 0 are obtained for the blanks (samples without heparin) and of pseudo-order 1 for the samples containing heparin.
The N-desulfation levels are determined taking into account the kinetic parameters for the samples (extrapolation of the curves and determination of the parameters for a reaction at 100% completion) and the values given by the standard at 100% N-desulfation, as in example 1.

The method above applies to nonhydrophobized N-desulfated heparin samples in the sodium salt form (and not in the cetyltrimethylammonium salt form).

c) Coupling of Cholic Acid

The primary amine functional groups (—NH₂) released on the heparin will be involved in a coupling reaction with the carboxylic acid (—COOH) functional group of the molecules of bile acid, resulting in the formation of a covalent bond of the amide (—CO—NH—) type. This reaction is preferably carried out with activation of the carbonyl group of cholic acid, given that the primary amine functional group is not very reactive.

Indeed, the carbon of the carboxylic acid (COOH) functional group is not sufficiently electrophilic to be able to be attacked by the electron doublet of the nitrogen atom. To make the carbon more electrophilic, acyl chlorides (R—CO—Cl) or acid anhydrides (R—CO—O—CO—R) for example are normally used. Another way is to use activated complexes. The chemistry of activating groups is thus widely used in peptide synthesis. In the context of the present invention, a coupling agent is preferably used to activate the terminal carboxylic functional group of the bile acid.

The coupling agent used to activate the terminal carboxylic functional group of the bile acid is preferably chosen from benzotriazolyloxytris(dimethyl-amino)phosphonium hexafluorophosphate (BOP), benzotriazolyloxytrispyrrolidinophosphonium hexafluorophosphate (PyBOP) and bromotrispyrrolidinophosphonium hexafluorophosphate (PyBroP).

For example, BOP comprises a good nucleofuge group (leaving group), HOBT (1-hydroxybenzotriazole), which has the advantage of accelerating the coupling reaction and of eliminating unwanted reactions; the oxyphosphonium salt constitutes the “coupling agent” part and binds to the carboxylate to activate the carbonyl (Evin 1978).

The chemical formula of BOP is the following:

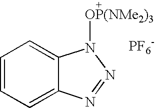

In the method developed in accordance with the invention, the coupling agent makes it possible to obtain the hydrophobized polysaccharide in a single step and thus avoids having to synthesize and isolate beforehand an activated ester of cholic acid which are two long and expensive steps.

The coupling reaction between the N-desulfated heparin and the bile acid is preferably carried out in an organic medium. Heparin is a very highly ionized polymer and is therefore highly soluble in water but insoluble in organic solvents. Cetyltrimethylammonium bromide has a long hydrocarbon chain of 16 carbon atoms and has surfactant properties because of its cationic polar head (quaternary ammonium) which confers its solubility in water on it. When a solution of this compound is poured into a heparin solution, the sodium ions of the heparin are then displaced and a salt forms between the acid functional groups and the ammonium functional groups. The hydrophobic salt of heparin thus obtained instantly precipitates since it is insoluble in water. The heparin in cetyltrimethylammonium salt form is then soluble in the organic solvents.

The method used may be the following, if the example is taken of a coupling with cholic acid.

First of all, the cetyltrimethylammonium salt of desulfated heparin should be allowed to dissolve in the hot state in chloroform at 60° C. and the following steps should be carried out during this period:
introduce the cholic acid into a small flask and solubilize it with dimethylformamide (DMF)
add 1.2 equivalents (relative to the acid) of a tertiary amine: N,N-diisopropylethylamine (DIEA)
add one equivalent of BOP and heat, with stirring, for a few minutes until all the BOP has dissolved
place 0.8 equivalent of DIEA in the flask containing the heparin
add the contents of the small flask (cholic acid) to the flask containing the heparin and add 0.3 equivalent of BOP
leave the whole stirring for at least one night at 60° C. and then evaporate all the chloroform
add ether in order to initiate the precipitation of the polymer and triturate in the ether
leave stirring in the ether until the agglomerates disintegrate into a fine powder
filter and rinse with ether; the crude product is obtained: heparin coupled with cholic acid but still in cetyltrimethylammonium salt form

Treatment of the Crude Material
take up in ethanol in the hot state (add some DMSO as required)
the crude product should be completely solubilized
with stirring in the hot state, add a saturated calcium chloride solution in ethanol, the hydrophobized heparin precipitates. This operation makes it possible to displace the cetyltrimethyl-ammonium salt
transfer the precipitated product into centrifugation tubes
triturate in hot ethanol, centrifuge. Repeat this operation of rinsing with ethanol 4 to 5 times. This operation makes it possible to eliminate the cetyltrimethylammonium chloride which is soluble in hot ethanol
combine the pellets by taking them up in hot DMSO
purification by successive dialyses against: DMSO/ethanol in the hot state, ethanol, DMSO/ethanol, ethanol, DMSO, water, water several times. At the end, the solution is white (translucent to opaque)
concentration and then freeze-drying. The hydrophobized heparin is obtained in the calcium salt form.

It is possible to obtain hydrophobized heparins with other counter-ions than calcium. Indeed, the applicant also obtained forms with magnesium and sodium. Now, the cetyltrimethylammonium salt cannot be properly removed from the heparin during treatment of the crude material with sodium chloride. The applicant thus observed that the reaction was complete when a bivalent cation (Ca⁺⁺ or Mg⁺⁺) is used.

Production of the Magnesium Form:

The crude material obtained from the coupling is treated with a saturated magnesium chloride solution in ethanol (in place of calcium chloride) and then subjected to an identical treatment to that described above.

Production of the Sodium Form:

The hydrophobized heparin final product in calcium salt form is used as starting material, solubilized in water. Phosphate buffer pH 7.4 is added thereto; the calcium binds to the phosphate ions to form an insoluble calcium phosphate precipitate. The hydrophobized heparin is then in the sodium form.

The preferred coupling level is that which makes it possible to obtain a heparin where the number of grafted molecules of bile acid per 100 disaccharide units of the heparin is between about 15 and about 80, preferably between about 20 and about 60.

With the use of an agent which makes it possible to activate the carboxylic functional group of the bile acid, it is possible to reach in general an amidation level of between 80% and 95%.

The coupling level (or the number of grafted molecules of bile acid per 100 disaccharide units of heparin) may be calculated by, determining the residual NH₂ level on a hydrophobized heparin in conformity with the invention.

For that, it is necessary beforehand, on a larger sample, to carry out the coupling of TNBS onto all the NH₂ groups of the heparin (a higher concentration of TNBS is used). The picric acid and the borate buffer are then removed by dialysis, and then a step of concentration and freeze-drying is carried out. The residual NH₂ level is determined by measuring the optical density at 420 nm for a solution of hydrophobized heparin coupled to TNBS.

d) Method of Incorporating Active Ingredients into the Nanoparticles of Hydrophobized Heparin

The incorporation of active ingredients into the vector consisting of the nanoparticles of hydrophobized heparin in accordance with the invention was carried out by direct dissolution.

An example of a method of incorporation by direct dissolution is described below. 30 mg of hydrophobized heparin are introduced into a tube and 3 ml of distilled water are added thereto. After dissolution, a large excess of active ingredient (15 to 30 mg) is introduced into the mixture.

A phase of stirring the mixture obtained is carried out by means of a magnetic stirrer bar for several days. The mixture is then centrifuged and the supernatant is passed through a 0.45 μm filter. The quantity of active ingredient solubilized in the solutions of hydrophobized heparin may be determined by UV/visible spectroscopy or by HPLC.

However, it is also possible to incorporate the active ingredient into the nanoparticles by dialysis, oil/water emulsion or solvent evaporation.

### FIGURES

FIG. 1: Variation of the blanks (samples without heparin) during assay of the amine functional groups with TNBS.

FIG. 2: Kinetics of the reaction for assaying the amine functional groups for a heparin N-desulfated at 100% with TNBS (experimental values and calculated values).

FIG. 3: Incorporation of Carbamazepine into heparin and HEP₁₉CHO in calcium salt form (Cp=8 mg/ml) after 3 days.

FIGS. 4 and 5: Incorporation of Carbamazepine (CBZ), Nifedipine (NIF) and Itraconazole (ITR) into HEP₁₉CHO (Cp=8.6 mg/ml) after 6 days. FIG. 4: Concentrations of active ingredient in water and in the hydrophobic domains. FIG. 5: Coefficient of partition between the hydrophobic domains and the aqueous phase.

FIG. 6: Incorporation of Nifedipine into various samples of modified heparin (Cp #8 mg/ml) after 4 days. Influence of the degree of hydrophobization and of the nature of the counter-ion.

FIG. 7: Absorption of Nifedipine by the everted intestinal sac for 90 minutes. Effect of vectorization by hydrophobized heparin (HEP30CHO) in relation to a control solution of Nifedipine (water/DMSO).

### EXAMPLE

### Example 1

### Determination of the Percentage N-desulfation of Two Samples of Hydrophobized Heparin

Method of Assay with TNBS

Solutions of N-desulfated heparin are prepared in borate buffer BB (0.1 M Na₂B₄O₇-10H₂O, pH10) (the quantities to be used are calculated so as to have a priori about 1.6 to 2.4 mM of NH₂). The solution of TNBS is diluted in water so as to obtain a concentration of 0.08 M. The solutions are distributed into tubes; each heparin sample is duplicated with a sample to which a dilution factor of 0.75 is applied (table 1).

TABLE 1
Distribution for assay of the amines with TNBS

| | Blanks | Heparin C1 | Heparin C2 |
|---|---|---|---|
| Heparin solution | 0 | 500 | 375 |
| BB | 500 | 0 | 125 |
| TNBS | 500 | 500 | 500 |

Blanks: Samples without heparin

The assays always include the reference samples ND₁₀₀-HEP (completely N-desulfated heparin).

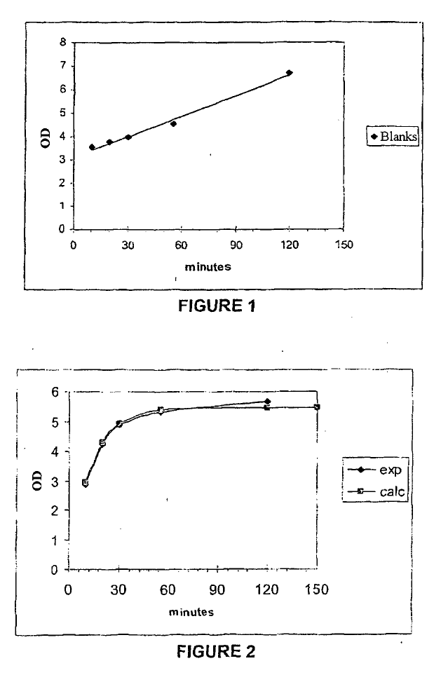

The time is counted from the addition of TNBS (the additions are made at regular intervals). The reaction occurs at room temperature. Quantities of 100 μl are collected at various times and immediately diluted by adding 800 μl of BB. The OD is read at 420 nm and the values are multiplied by 9 for the calculations. The OD values of the blanks collected at the corresponding times are subtracted from the OD values obtained for the heparin samples. The variation of the blanks is linear (FIG. 1).

The kinetic parameters for the heparins are calculated so as to be able to determine the theoretical OD (ODmax) for 100% completion of the assay reaction. The concentration of TNBS being much higher than the concentration of NH₂, the former may be considered as a constant (it will be expressed by) and the kinetics is then of the pseudo-order 1 type. The rate V of the assay reaction may be considered as being directly proportional to the concentration of the NH₂ groups:
V=k··[NH₂]

The variation of the OD values follows the following law:
OD=ODmax·(1−e^{-k·t})

It is possible to linearize this equation in the form Y=a+b·t, by calculating the logarithm of the difference between a theoretical ODmax and the experimental OD at the time t. This gives:

Y=Ln(ODmax−ODt), and by plotting Y=f(t), a straight line is obtained with the transformed experimental data for which the ordinate at the origin and the slope are respectively:
a=Ln(ODmax) and b=−k.

The experimental data are therefore transformed (table 2) using a theoretical ODmax for which the value is adjusted so as to obtain the best coefficient of determination for the straight line Y=f(t) and the smallest differences between the experimental values and the calculated values which it is possible to calculate and to visualize by the superposition of the theoretical curve with the experimental data (FIG. 2). This adjustment is very easy to carry out having entered the data into a model calculation sheet

(Microsoft® Excel spreadsheet).

TABLE 2
Transformation of the experimental data for
the assay of ND₁₀₀-HEP

| | Time (min) | | | | |
|---|---|---|---|---|---|
| | 10 | 20 | 55 | 120 | ODmaxTheo |
| ODt (exp.) | 2.880 | 4.086 | 4.437 | 4.437 | 4.4444 |
| Ln (ODmaxTheo- | 0.448 | −1.026 | −4.906 | −4.906 | |
| ODt) | | | | | |

The parameters calculated are the following:

TABLE 3
Kinetic parameters for the assay of ND₁₀₀-HEP
with TNBS

| b (slope) | a (origin) | r² |
|---|---|---|
| −0.11685433 | 1.4825799 | 0.9968 |
| ODMax = 4.404 | | |
| t_{1/2} = | 5.93 min | |
| t_{99%} = | 39.41 min | |

These calculations are performed for the various ND-HEP samples used in an assay in order to determine the calculated ODMax for each of them. For some, depending on the concentration of NH₂ effectively present, the kinetics are not totally complete in one hour. The method demonstrates its value in this case since it makes it possible to draw a theoretical curve through the experimental points and to calculate the ODMax which would be obtained if all the NH₂ groups had reacted.

Assuming that the molar extinction coefficient is the same for all the compounds, it is possible to determine the percentage NH₂ according to the formula:

$$\% {NH}_{2} = \frac{Conc . {ND}_{100} - HEP}{{OD}_{ND 100 - HEP}} \times \frac{ODx}{Conc . X}$$

The ODMax values and the %NH₂ for ND₃₀-HEP and ND₆₃-HEP are presented by way of example (table 4).

TABLE 4
Calculation of the % NH₂ for two ND-HEP
derivatives

| Name | C (mg/ml) | ODMax | % NH₂ |
|---|---|---|---|
| ND₁₀₀-HEP | 0.835 | 4.404 | 100% |
| ND₆₃-HEP | 1.68 | 5.619 | 63.4% |
| ND₃₀-HEP | 2.43 | 3.860 | 30.1% |

### Example 2

### Determination of the Increase in Aqueous, Solubility of Some Water-insoluble Active Ingredients

Trials for incorporation of active ingredients were carried out by stirring the molecule to be tested, in the solid state, in a solution of heparin hydrophobized with cholic acid, the heparin having been N-desulfated at 19%, and prepared in calcium salt form (HEP19CHO), at the Cp concentration (mg/ml) for several days. The nonsolubilized active ingredient is removed after filtration of the solutions on a membrane having a porosity of 0.45 μm. The quantity of active ingredient in solution is then determined. Having determined the quantities solubilized in water and those dissolved in the hydrophobic domains, it is then possible to calculate a partition coefficient hydrophobic domains/water which is called Log P′ by analogy with Log P.

Indeed, the relative solubility of molecules between an oily phase and an aqueous phase is described by the partition coefficient P which represents the distribution of the molecules between two solvents, generally water and octanol. The use of its logarithm, log P, is often preferred. Hence log P>0 for the hydrophobic molecules and log P<0 for the hydrophilic molecules. Thus, when Log P is equal to 3, that means that the ratio of the solubilities of the active agent in water and in the oily phase is equal to 1000. Likewise for Log P′ which expresses the ratio of the solubilities of the active agent in water and in the solution of hydrophobized heparin.

The following molecules were tested (see table 5).

TABLE 5
Incorporation of hydrophobic molecules into a
heparin hydrophobized with cholic acid (19%
N-desulfation at Cp = 8 mg/ml) in calcium salt form
(HEP19CHO)

| | Quantity of | Quantity of | |
|---|---|---|---|
| | active | active | |
| | ingredient | ingredient | |
| Active | solubilized | solubilized | |
| ingredient | (μmol/g) | (mg/g) | Log P′ |
| Yellow OB | 14 | 3.75 | 5 |
| Itraconazole | 180 | 130 | 3.32 |
| Progesterone | 242 | 76 | 1.89 |
| Nifedipine | 260 | 90 | 2.25 |
| Carbamazepine | 524 | 124 | 1.0 |

These results show that the presence of heparin molecules having hydrophobic groups makes it possible to significantly increase the solubility of molecules that are weakly soluble in water. Among the molecules tested, carbamazepine is that which exhibits the highest affinity for the hydrophobic domains. Molecules such as itraconazole and nifedipine, whose solubility in water is extremely low, exhibit an increased solubility when they are incorporated into the hydrophobized heparin, which is all the more advantageous since they are active at a low dose. Thus, nifedipine and itraconazole are respectively 175 and more than 2000 times as soluble in a solution of hydrophobized heparin as in water.

These results show that the hydrophobic domains of heparin modified with cholic acid make it possible to incorporate hydrophobic active ingredients and to considerably increase their solubility in water.

### Example 3

### Incorporation of Active Ingredients into an Aqueous Solution of Hydrophobized Heparin

The incorporation of active ingredients was essentially carried out by the dissolution method. In this method, the compound to be incorporated is placed in solid form in a tube containing water or a colloidal solution of hydrophobized heparin, at the concentration Cp (mg/ml).

The mixing is performed with stirring at room temperature for various times. The compound to be incorporated remains at saturating concentration in the medium. At the end of the incorporation period, the tubes are subjected to a centrifugation step and the supernatants are filtered on 0.45 μm filters and assayed. Control solutions (water or nonhydrophobized heparin) are treated in the same manner.

The quantity of active ingredient incorporated into the various media is quantified by HPLC and/or by measuring the UV absorbance.

The filtration and the determination of the quantity dissolved in water then make it possible to obtain the quantity of active ingredient incorporated into the hydrophobic domains of the hydrophobized heparins in accordance with the invention, and to calculate the Log P′ as defined above.

Thus, the incorporation is expressed in milligram of active ingredient per gram of polymer (mg/g P) or in micromole of active ingredient per gram of polymer (μmol/g P). This incorporation level is calculated by taking the ratio between the concentration of the active ingredient in solution and the concentration of the polymer. It is important to take into consideration the quantity of active ingredient solubilized in controls such as water or a solution of nonhydrophobized heparin (native heparin or N-desulfated sample). The difference in concentration of active ingredients in water and in a solution of hydrophobized heparin represents the quantity of active ingredient effectively present in hydrophobic micro-domains formed by the grouping together of cholic acid residues.

a) Carbamazepine

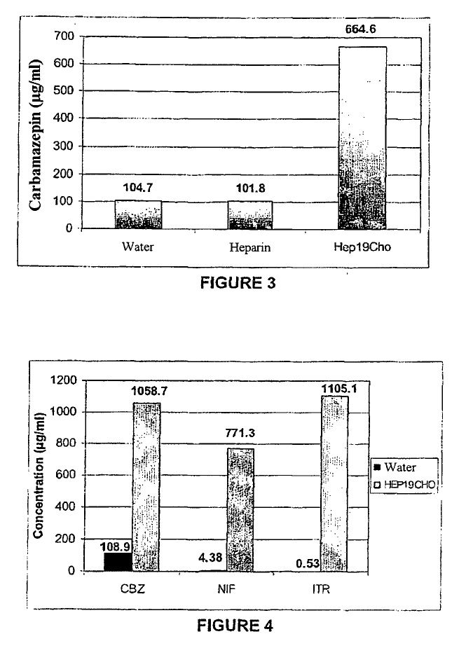

FIG. 3 represents the level of incorporation of carbamazepine into the two control solutions (unmodified heparin and water) and in a solution at 8 mg/ml of heparin hydrophobized with cholic acid whose N-desulfation level is 19% (HEP₁₉CHO) in accordance with the present invention.

These results show that the concentration of active ingredient in solution in water or in the presence of heparin is very markedly less than the concentration in a solution comprising hydrophobized heparin in accordance with the invention. In this example, the gain in solubility relative to water exceeds 500%.

It is possible to determine the concentration of carbamazepine present in the hydrophobic domains (559.9 mg/ml) by subtracting the concentration of the active ingredient present in the aqueous phase from the concentration of carbamazepine in the HEP₁₉CHO solution. The level of incorporation into the hydrophobic domains is then determined by dividing the concentration of active ingredient incorporated by the concentration by mass of the polymer in solution. There is thus obtained for carbamazepine in HEP₁₉CHO according to the conditions mentioned in Error! Source of the reference not found. an incorporation level of 70 mg/g P.

b) Other Hydrophobic Active Ingredients

Incorporation trials under similar conditions to those described in example 3 for carbamazepine (CBZ) were performed for two other hydrophobic active ingredients Itraconazole (ITR) and Nifedipine (NIF).

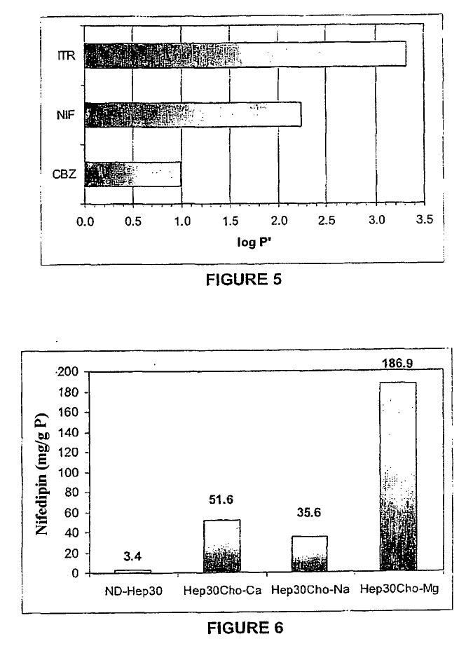

FIG. 4 represents the quantities of CBZ, NIF and ITR present in water and in the hydrophobic domains of a heparin hydrophobized. with cholic acid at an N-desulfation level of 19% (HEP₁₉CHO) after 6 days of incorporation period. Under these conditions, the quantity of CBZ in solution is increased ten fold, those of NIF and ITR were increased by more than 175-and 2000-fold respectively. The partition coefficients of these three molecules are greater than or equal to 1, inducing a very high affinity in favor of the hydrophobic domains (FIG. 5).

### Example 4

### Influence of the Counter-ion

A difference in solubilizing power of the hydrophobic domains according to the nature of the counter-ion of the heparin part of the vectors was also demonstrated. Thus, in the case of nifedipine, the bivalent cations allow the incorporation of more active ingredient than a monovalent cation such as sodium (FIG. 6).

In the case of the sodium forms, the negative charges of the heparin are individualized. Neutralization of these charges by a bivalent cation promotes the grouping together of the hydrophilic chains, allowing easier assembly of the hydrophobic domains. The putting in place of the hydrophobic domains is therefore promoted by the presence of bivalent cations. Thus, it can be assumed that Mg²+induces a different arrangement of the hydrophilic crown which results in an impact on the structure of the domains, causing them either to come closer together, or facilitating the formation of larger domains capable of receiving more host molecules.

For smaller quantities of Nifedipine, a high incorporation of the active ingredient is observed in the case of HEP₃₀CHO—Ca compared with HEP₃₀CHO—Na.

### Example 5

### Determination of the Efficiency of Coupling Between Heparin and Cholic Acid (HEPCHO)

To evaluate the coupling efficiency (or level), the number of residual amine functional groups was quantified at the end of the hydrophobization reaction with cholic acid. The applicant used a method which makes it possible to couple TNBS to the amine functional groups of the HEPCHOs in order to isolate the compounds obtained and to subsequently quantify their absorption.

Labeling of HEPCHO with TNBS

The procedure used is adapted from the TNBS-based assay protocol using higher concentrations of TNBS and sufficient quantities of heparin so as to allow the treatment of the, final product for its characterization.

The reaction was carried out on HEP₃₀CHO (heparin N-desulfated at a level of 30% and then hydrophobized with cholic acid), HEP₆₃CHO (heparin N-desulfated at a level of 63% and then hydrophobized with cholic acid) and on the respective original N-desulfated heparins ND₃₀-HEP and ND₆₃-HEP.

The procedure is very simple. It consists in reacting TNBS with a quantity of about 100 mg of HEPCHO or of ND-HEP dissolved in borate buffer at pH 10. At the end of the reaction, the appearance of the solutions is in agreement with the intensity of the expected labelings: the HEPCHOs are orange and the ND-HEPs red (ND₆₃-HEP is particularly intense red).

The contents of the tubes are then dialyzed for purification. Indeed, the complete elimination of the picric acid in excess should be ensured. The compounds obtained are trinitrophenylamine type derivatives designated ND-HEP-TNP and HEPCHO-TNP. Following this purification step, the volume of the solutions is accurately adjusted for measurement of the optical density at 420 nm. The samples are then concentrated and freeze-dried.

Characterization of the Labeling and Determination of the Coupling Efficiency

The absence of residual picric acid in the four samples was first verified. Aqueous solutions of heparins labeled with TNBS were therefore injected by HPLC. Control solutions containing picric acid or heparin-TNP/picric acid mixtures were also injected. The HPLC chromatograms show the absence of residual picric acid from all the HEPCHO-TNP and ND-HEP-TNP samples.

The efficiency of coupling of cholic acid to the NH₂ functional groups was determined according to two methods: measurement of the ODs at the end of the dialysis step and analysis from the freeze-dried finished product.

At the end of the final dialysis, the volumes of the purified solutions of heparins labeled with TNBS were accurately adjusted to 50 ml for reading of the OD at 420 nm. The ND-HEP-TNP type solutions, which are 15 more intensely colored, were the subject of a 1/10 dilution because of their excessively intense coloration. This first evaluation of the coupling efficiency is based on a postulate which attributes 63% of NH₂ titer to the ND₆₃-HEP-TNP product from which the NH₂ contents of the other compounds are deduced as a function of their OD to heparin-TNP concentration ratios (table 6).

TABLE 6
Evaluation of the percentage of amine and of
the efficiency of the coupling of cholic acid to
heparin-TNPs, at the end of the reaction with TNBS

| | Weighings* | Conc. | OD | | | |
|---|---|---|---|---|---|---|
| Name | (mg) | (mg/ml) | (420 nm) | OD/C | % NH₂ | Efficiency |
| ND₆₃-HEP- | 115.8 | 0.232 | 0.935 | 4.04 | 63% | 93.5% |
| TNP | | | | | | |
| HEP₆₃CHO- | 103 | 2.06 | 0.537 | 0.26 | 4.07% | |
| TNP | | | | | | |
| ND₃₀-HEP- | 105 | 0.210 | 0.457 | 2.18 | 33.96% | 81.0% |
| TNP | | | | | | |
| HEP₃₀CHO- | 102.9 | 2.06 | 0.852 | 0.41 | 6.46% | |
| TNP | | | | | | |

*initial weight of heparin used in the coupling reaction

Thus, on the basis of this postulate, a percentage of primary amine functional groups of about 34% is found for the ND₃₀-HEP-TNP compound. This value is quite similar to the value determined by the colorimetric method with TNBS on the ND₃₀-HEP sample (30.1%) which makes it possible to accept the values calculated for HEP₆₃CHO-TNP and HEP₃₀CHO-TNP. The method also indicates that about 94% of the NH₂ functional groups of ND₆₃-HEP were effectively substituted with cholic acid. The coupling reaction on ND₃₀-HEP made it possible to affect only 81% of the amine functional groups initially present.

The second method for evaluating the efficiency of coupling of cholic acid involves the preparation of solutions from the four freeze-dried samples, followed by a reading of the optical density at 420 nm. The calculations are different and take into account the determination of the molar concentration of NH₂ based on the OD and a molar extinction coefficient ε=4700 M⁻¹.cm⁻¹. A ratio is then calculated between this molar concentration and the concentration by mass corrected by the contribution by mass of the substitution with the TNP group (n/C_{cor}). Knowing the theoretical ratio n/C for the heparin ND₁₀₀-HEP (n/C=1.49×10⁻mol/g), it is then possible to calculate the percentage of NH₂ for the samples (table 7). The calculation of the coupling efficiency takes in particular into account the number of residues modified with cholic acid and the contribution by mass made by these groups.

TABLE 7
Evaluation of the percentage of amine and of
the efficiency of the coupling of cholic acid to
heparin-TNPs isolated and redissolved in solution

| | | | No. | | | |
|---|---|---|---|---|---|---|
| | | | mol/l | | | |
| | Conc | OD | (n) | | | |
| Name | (mg/ml) | (420 nm) | NH₂ | n/C_{cor} | % NH₂ | Efficiency |
| ND₆₃-HEP-TNP | 0.037 | 0.141 | 3.00 × | 9.72 × | 65.2% | 91.16% |
| | | | 10⁻⁵ | 10⁻⁴ | | |
| HEP₆₃CHO-TNP | 0.250 | 0.074 | 1.57 × | 6.38 × | 4.3% | |
| | | | 10⁻⁵ | 10⁻⁵ | | |
| ND₃₀-HEP-TNP | 0.112 | 0.253 | 5.38 × | 5.35 × | 35.9% | 79.15% |
| | | | 10⁻⁵ | 10⁻⁴ | | |
| HEP₃₀CHO-TNP | 0.950 | 0.419 | 8.91 × | 9.57 × | 6.4% | |
| | | | 10⁻⁵ | 10⁻⁵ | | |

The percentage of amine functional groups thus determined is also quite similar to the values found for the calorimetric assay carried out on the ND-HEP samples. Likewise, this method of indirect determination gives efficiencies of coupling of cholic acid to N-desulfated heparins close to those determined by the preceding method (at the end of the purification by dialysis).

From these data, it is possible to have an idea of the structure of the modified heparins according to the following reasoning:

If it is considered that the heparin used has on average per glucosamine unit:
2% of NH₂ functional groups (calorimetric assay with TNBS);
15 to 16% of N-acetyl functional groups (H NMR spectra);
83% of N-sulfate functional groups (by difference).

The glucosamine unit of our reference ND₁₀₀-HEP is at “100% NH₂” (it is on this basis that all the other % NH₂ of ND-HEPₓ are determined), that is to say in reality about 15% of N-acetyl functional groups and 85% of NH₂ functional groups.

Thus, the glucosamine units of the compounds HEP₆₃CHO and HEP₃₀CHO have the following theoretical structure (table 8).

TABLE 8
Structures of the glucosamine units of two
heparins hydrophobized with cholic acid

| | | HEP₆₃CHO | HEP₃₀CHO |
|---|---|---|---|
| | % NH₂relative | 4.3% | 6.4% |
| | to ND₁₀₀-HEP | | |
| Substituent | NH₃⁺ | 3.7% | 5.4% |
| on G-2 | | | |
| | —NH-cholyl | 51.7% | 25.1% |
| | —NH—SO₃⁻ | 29.6% | 54.5% |
| | —NH—CO—CH₃ | 15% | 15% |

The percentage corresponding to —NH-cholyl represents the number of cholic acid molecules grafted per 100 disaccharide units of heparin.

### Example 6

### Determination of the Increase in Intestinal Absorption in the Presence of Hydrophobized Heparin

The applicant demonstrated on an animal model the modification of the intestinal absorption of certain sparingly water-soluble active ingredients in the presence of heparin hydrophobized with cholic acid, the heparin having been N-desulfated at 30%, and prepared in magnesium salt form (HEP30CHO).

The model used is the rat everted intestinal sac. It is an ex vivo method on a portion of isolated organ (Barth et al. 1998, 1999). To do this, the small intestine of adult rats is removed and then turned inside out by means of a glass rod. Sacs about 2 cm in length are made by hermetically closing the ends of the intestinal segments. Said sacs then have the mucous side comprising the intestinal villosities on the outside. These sacs are incubated in an oxygenated cell culture medium at 37° C. that is rich in vitamins and nutrients (TC 199) so as to increase the survival of the intestinal cells. Under these conditions, the intestinal mucosa is physiologically functional since the cells consume the glucose in the culture medium and produce an abundant mucus during the experiment. The active ingredient whose absorption it is desired to measure is placed in a solution outside the sac. The sacs are collected at various times and the quantity of active ingredient which is absorbed by the intestinal mucosa is quantified inside the sacs by HPLC.

Two experiments were carried out on this model in the presence of nifedipine vectorized by hydrophobized heparin in accordance with the invention. Given the very low solubility of nifedipine in aqueous solution, the control solutions were made from nifedipine solubilized in DMSO (dimethyl sulfoxide) and added to the cell culture medium (that is 0.1% of DMSO maximum) so as to obtain a homogeneous solution of nifedipine.

To prepare the media containing hydrophobized heparin, a quantity of polymer corresponding to 20-25 μg/ml of active ingredient is dissolved in water.

The absorption of nifedipine is quantified at the end of 30, 60 and 90 minutes of incubation.

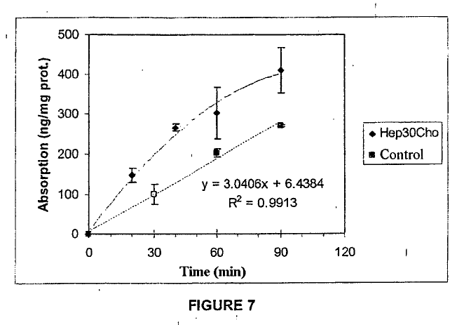

The results of this experiment are represented in FIG. 7.

FIG. 7 represents the absorption of nifedipine by the intestinal mucosa as a function of its vectorization or otherwise. The difference in the rate of absorption between the nifedipine vectorized by HEP₃₀CHO and a solution comprising this active ingredient and DMSO is obvious.

Thus, for similar concentrations of nifedipine, vectorized by heparin or solubilized by DMSO, the applicant was able to demonstrate a true promoter effect of the absorption in the case of the hydrophobized heparin. Indeed, the rate of absorption of nifedipine vectorized by the polymer is markedly greater than that of the nifedipine kept in solution by means of DMSO. Without the artifice of increasing the solubility of nifedipine with DMSO, the quantities of this active ingredient in the control sacs would have been very low. The promoter effect of absorption is therefore considerably higher than what FIG. 7 shows.

These results make it possible to envisage the intensification of the intestinal absorption and consequently the increase in the bioavailability in vivo in nifedipine and other active ingredients vectorized by this type of polymer.

Thus, the present invention makes it possible to provide a new vector which makes it possible to significantly increase the solubility and the intestinal absorption of lipophilic active ingredients that are normally weakly absorbed by the cells of the intestinal mucosa, such as medicaments belonging to the class of anticancer agents or anti-inflammatory agents for example.

Furthermore, the nanoparticles in accordance with the invention can be easily integrated into a galenic carrier traditionally used for oral administration of medicaments, such as granules, microgranules, tablets, gelatin capsules or solutions to be taken orally.

### Bibliographic References

Akiyoshi, K., S. Deguchi, N. Moriguchi, S. Yamaguchi and J. Sunamoto (1993). Self-aggregates of hydrophobized polysaccharides in water. Formation and characteristics of nanoparticles. Macromolecules, 26, 3062-3068.

Barthe, L., J. F. Woodley and G. Houin (1999). Gastrointestinal absorption of drugs: methods and studies. Fundamental and Clinical Pharmacology, 13, 154-168.

Barthe, L., J. F. Woodley, S. Kenworthy and G. Houin (1998). An improved everted gut sac as a simple and accurate technique to measure paracellular transport across the small intestine. European Journal of Drug Metabolism and Pharmacokinetics, 23, 313-323.

Desai, M. P., V. Labhasetwar, G. L. Amidon and R. J. Levy (1996). Gastrointestinal uptake of biodegradable microparticles: effect of particle size. Pharmaceutical Research, 13, 1838-1845.

Diancourt, F., C. Brand and M. Vert (1996). Chemical modifications of heparin. II. Hydrophobization of partially N-desulfated heparin. Journal of bioactive and compatible polymers, 11, 203-218.

Evin, G. (1978). Le BOP, nouveau réactif de couplage peptidique. Applications à la synthèse de la Somatostatine et de dérivés solubles de la Pepstatine A. [BOP, novel peptide coupling reagent. Applications to the synthesis of somatostatin and soluble derivatives of pepstatin A.] Nancy, Université de Nancy I: 200.

Florence, A. T. (1997). The oral absorption of micro- and nanoparticulates: neither exceptional nor unusual. Pharmaceutical Research, 14, 259-266.

Inoue, Y. and K. Nagasawa (1976). Selective N-desulfation of heparin with dimethyl sulfoxide containing water or methanol. Carbohydrate Research, 46, 87-95.

Larhed, A. W., P. Artursson, J. Graasjö and E. Björk (1997). Diffusion of drugs in native and purified gastrointestinal mucus. Journal of Pharmaceutical Sciences, 86, 660-665.

Nagasawa, K. and Y. Inoue (1974). Solvolytic desulfation of 2-deoxy-2-sulfoamino-D-glucose and D-glucose 6-sulfate. Carbohydrate Research, 36, 265-271.

O'Hagan, D. T. (1996). The intestinal uptake of particles and the implications for drug and antigen delivery. Journal of Anatomy, 189, 477-482.

Park, K. and R. Robinson (1984). Bioadhesive polymers as platforms for oral controlled drug delivery: method to study bioadhesion. International Journal of Pharmaceutics, 19, 107-127.

Ponchel, G., M. J. Montisci, A. Dembri, C. Durrer and D. Duchêne (1997). Mucoadhesion of colloidal particulate systems in the gastro-intestinal tract. European Journal of Pharmaceutics and Biopharmaceutics, 44, 25-31.

Snyder, S. L. and P. Z. Sobocinski (1975). An improved 2,4,6-trinitrobenzenesulfonic acid method for the determination of amines. Analytical Biochemistry, 64, 284-288.

1. An amphiphilic heparin derivative formed from an at least partially N-desulfated heparin and from at least one bile acid, comprising one or more bile acid molecules grafted onto the heparin molecule by an amide bond formed between the terminal carboxylic acid functional group of the bile acid and a primary amine functional group of the heparin, originally present in the heparin or resulting from the N-desulfation, wherein the number of bile acid molecules grafted per 100 disaccharide units of the heparin is between about 15 and about 80.

2. The amphiphilic heparin derivative as claimed in claim 1, wherein the number of bile acid molecules grafted per 100 disaccharide units of the heparin is between about 20 and about 60.

3. The amphiphilic heparin derivative as claimed in claim 1, wherein the bile acid is selected from the group consisting of cholic acid, deoxycholic acid, lithocholic acid, cholanic acid and chenodeoxycholic acid, and mixtures thereof.

4. The amphiphilic heparin derivates as claimed in claim 1, wherein said amphiphilic heparin derivative is prepared in calcium, magnesium or sodium salt form.

5. The amphiphilic heparin derivates as claimed in claim 1, wherein said amphiphilic heparin derivatives are capable of spontaneously assembling in an aqueous medium to form nanoparticles.

6. Nanoparticles which can be formed from the amphiphilic heparin derivative as claimed in claim 1.

7. The nanoparticles as claimed in claim 6, wherein said nanoparticles have an average size of between 10 nm and 1 μm.

8. The nanoparticles as claimed in claim 6, wherein said nanoparticles contain one or more inner hydrophobic domains and a hydrophilic outer surface.

9. The nanoparticles as claimed in claim 6, wherein said nanoparticles additionally contain one or more hydrophobic active ingredients dissolved in its hydrophobic inner domain.

10. The nanoparticles as claimed in claim 9, wherein said active ingredients additionally carry one or more polar groups.

11. The nanoparticles as claimed in claim 9, wherein said active ingredients are selected from the group consisting of anti-inflammatory agents, antifungal agents, calcium channel inhibitors and anticancer agents.

12. Vectors for active ingredients which can be administered by the oral route comprising the nanoparticle as claimed in claim 9.

13. Vectors for active ingredients which make it possible to increase the absorption of said active ingredients by the intestinal mucosa comprising the nanoparticle as claimed in claim 9.

14. Vectors for active ingredients which allow the gradual release of said active ingredients in the intestinal mucosa comprising the nanoparticle as claimed in claim 9.

15. The nanoparticles as claimed in claim 6, wherein said nanoparticles are in freeze-dried form.

16. A colloidal suspension in aqueous medium containing the nanoparticles as claimed in claim 6.

17. A pharmaceutical composition comprising the nanoparticles as claimed in claim 9, combined with at least one pharmaceutically acceptable excipient.

18. The pharmaceutical composition as claimed in claim 17, wherein said excipient is chosen to allow administration of active ingredients by the oral route.

19. The pharmaceutical composition as claimed in claim 18, wherein said composition is in the form of granules, microgranules, tablets, gelatin capsules or solutions to be taken orally.

20. A method for preparing the amphiphilic heparin derivative as claimed in claim 1, comprising the at least partial N-desulfation of a heparin, and then a coupling step which consists of reacting at least one primary amine functional group of the heparin, originally present or resulting from the N-desulfation, with the terminal carboxylic acid functional group, optionally in activated form, of at least one bile acid.

21. The method for preparing the amphiphilic heparin derivative as claimed in claim 20, wherein the coupling agent used to activate the terminal carboxylic functional group of the bile acid is selected from the group consisting of benzotriazolyloxytris(dimethylamino)phosphonium hexafluorophosphate (BOP), benzotriazolyloxytrispyrrolidinophosphonium hexafluorophosphate (PyBOP) and bromotrispyrrolidinophosphonium hexafluorophosphate (PyBroP).

22. A method for preparing the nanoparticles as claimed in claim 9, wherein the active ingredient is incorporated into said nanoparticles by direct dissolution with stirring, by dialysis, by oil/water emulsion or by solvent evaporation.

23. A method for increasing the solubility of a hydrophobic active ingredient in an aqueous medium comprising incorporating said active ingredient into the nanoparticle as claimed in claim 9.
